(19) United States
(12) Patent Application Publication
(10) Pub. No.: US 2007/0178141 A1
(43) Pub. Date: Aug. 2, 2007

(54) Vitamin B12 compositions

(75) Inventor: Chad Brown, Newport Beach, CA (US)

Correspondence Address: HELLER EHRMAN LLP, 1717 RHODE ISLAND AVE, NW, WASHINGTON, DC, 20036-3001, US

(73) Assignee: BEBAAS, INC., Newport Beach, CA (US)

(21) Appl. No.: 11/219,794

(22) Filed: Sep. 7, 2005

(51) Int. Cl. A61K 31/714; A61K 9/68; A61K 9/127; A61K 9/48; A61K 9/20

(52) U.S. Cl. 424/440; 514/52; 424/451; 424/450; 424/464

(57) ABSTRACT

The present invention provides improved vitamin B₁₂ compositions containing a mixture of vitamin B₁₂ analogues that are better absorbed into the bloodstream of a patient with vitamin B₁₂ deficiency, preferably via their mucosal membranes and also discusses convenient and inexpensive delivery methods for these compositions without the discomfort of subcutaneous or intramuscular injections.

### BACKGROUND OF THE INVENTION

1. Field of the Invention

The present invention provides improved vitamin B₁₂ compositions containing a mixture of vitamin B₁₂ analogues in effective amounts for enhanced delivery via the mucosal membranes, such as the mouth, nose, etc., to ameliorate any condition associated with vitamin B₁₂ deficiency in a patient.

2. Description of the Related Art

Vitamin B₁₂ is synthesized by microbes, but not by humans or plants. Gastrointestinal absorption of vitamin B₁₂, from food or supplements, depends on the presence of sufficient intrinsic factor and calcium ions. Adenosylcobalamin and methylcobalamin are the active forms of vitamin B₁₂ in humans. Vitamin B₁₂ deficiency may result from intrinsic factor deficiency (pernicious anemia), partial or total gastrectomy, or diseases of the distal ileum, intestinal problems and nerve damage, etc. People or patients who have conditions that make them vulnerable to vitamin B₁₂ deficiency include those with Crohn's disease, multiple sclerosis, HIV/AIDS, people who are 65 years or older, those with chronic intestinal inflammation, patients who have undergone intestinal surgery, patients whose food move too quickly throughout the intestine, people on strict vegetarian diets, people who drink alcohol excessively for longer than 2 weeks, people using acid reducing drugs for a long time period or patients that use chemotherapy drugs.

Vitamin B₁₂ has also been used in the treatment of IgE-mediated allergic diseases, such as allergic rhinitis and asthma. Oral ingested Vitamin B₁₂ is ineffective in the treatment of allergic disease, possibly due to liver metabolism.

Cyanocobalamin (Crystamine, Cyomin, Crysti 1000, Nascobal®) is the most widely sold analogue of Vitamin B₁₂. Cyanocobalamin is available in injectable (subcutaneous or intramuscular) and oral forms and has the advantage of having a stable shelf life at standard temperature and pressure (STP). Cyanocobalamin—Nascobal® is also available as an intranasal gel and has been clinically shown to maintain adequate serum levels of Vitamin B₁₂. The nasal gel can be self-administered through a simple nasal delivery system that avoids the discomfort of intramuscular injections of B₁₂.

Since vitamin B₁₂ is very large, orally ingested cyanocobalamin is improperly digested and only small amounts of the vitamin get absorbed by the host. The drawback of the injectable form is that it is invasive, expensive and inconvenient. Hence, there is a need for more effective forms of vitamin B₁₂ that can be absorbed more easily to ameliorate conditions associated with vitamin B₁₂ deficiency.

### SUMMARY OF THE INVENTION

The present invention provides compositions and convenient methods for the delivery of vitamin B₁₂ compositions to patients with vitamin B₁₂ deficiencies.

In one aspect, the present invention provides a pharmaceutical composition comprising an effective amount of a mixture of methylcobalamine, hydroxycobalamin, and cyanocobalamin and a pharmaceutically acceptable carrier.

In another aspect, the present invention provides a pharmaceutical composition wherein said composition is in the form of a gel, a sublingual lozenge, a solution suitable for aerosolization for mucosal, nasal or pulmonary administration, a liposomal formulation, fine particles in a form suitable for aerosolization for mucosal or pulmonary administration or a tablet.

In one embodiment, the pharmaceutical composition in the form of a gel is disposed within a capsule.

In yet another aspect, the present invention provides an apparatus for producing aerosolized doses of a composition in the form of fine particles, for mucosal or pulmonary administration, said apparatus comprising a means for introducing a preselected amount of said composition into a predetermined volume of a flowing propellent to produce an aerosolized quantity of said composition.

In a further aspect, the present invention provides a method of treating or ameliorating a disease associated with vitamin B₁₂ deficiency, comprising administering to a subject suffering from or suspected of suffering from said disease an effective amount of any of the compositions described above.

In one embodiment, the subject suffers from or is suspected to suffer from an anemia. In a further embodiment, the anemia could be due to pernicious anemia, drug administration to said subject, Crohn's disease or burns.

In another aspect, the present invention provides a method of treating or ameliorating a disease or syndrome in a subject, comprising administering to said subject an effective amount of any of the compositions defined above, wherein said disease or syndrome is selected from the group consisting of cobalamin malabsorption, a neurological disorder, atrophic body gastritis, an autoimmune disorder, and symptoms associated with therapy with gastric acid inhibitors or biguanides.

In one embodiment, the neurological disorder is selected from the group consisting of Alzheimer's disease, amyotrophic lateral sclerosis, multiple sclerosis, and ataxia.

In another aspect, the present invention provides a method of treating an inflammatory disease in a subject, comprising administering to said subject an effective amount of any of the compositions defined above.

In one embodiment, the inflammatory disease is joint inflammation or arthritis.

In yet another aspect, the present invention provides a method of reducing immunoglobulin E production in a subject comprising administering to said subject an effective amount of any of the compositions defined above.

In a further aspect, the present invention provides a method of treating or ameliorating a disease associated with vitamin B₁₂ deficiency comprising using an apparatus for producing aerosolized doses of the composition described above, to administer an effective dose of said composition to a subject suffering from or suspected of suffering from said disease.

In another aspect, the present invention provides a kit comprising a pharmaceutical composition comprising an effective amount of a mixture of methylcobalamine, hydroxycobalamin, and cyanocobalamin and a pharmaceutically acceptable carrier.

### DETAILED DESCRIPTION

Definitions

The following terms used in the specification and claims shall have the following meanings for the purpose of the Application.

By “active ingredients or compounds” of the invention are meant types of cobalamins including, but not limited to, methylcobalamine, cyanocobalamin, , hydroxycobalamin, etc. The active ingredients are used in different mixtures containing varying effective amounts of each active compound of the invention, which would be suitable for the treatment of different types of vitamin B₁₂ deficiencies.

By “mixture” is meant a combination containing different types of active ingredients defined above, in effective amounts, useful for the treatment of vitamin B₁₂ deficiency.

By “effective amount” is meant that amount, which when administered, either alone or in a mixture, is sufficient to effect the treatment of a condition with vitamin B₁₂ deficiency.

By “inert ingredients” is meant components like pharmaceutically acceptable carriers, adjuvant, diluents or excipients, etc., that must be compatible with the other ingredients of the formulation and not deleterious to the recipient thereof.

The term “composition” or a “formulation” as used herein is intended to encompass a product comprising the specified active ingredients in the specified amounts, as well as any product which results, directly or indirectly, from the combination of the specified active ingredients in the specified amounts. Such term is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing any active compound of the present invention and a pharmaceutically acceptable carrier.

The terms “administration of” and or “administering a” compound should be understood to mean providing any active compound of the invention, in any formulation, to an individual in need of treatment.

In accordance with the invention, vitamin B₁₂ is instilled in a carrier matrix, such as controlled release lozenges, troches, tablets, hard or soft capsules, syrups or elixirs, pressed pills, gel caps, chewing gum, gels such as metered gels that can be administered intranasally, nasal drops, creams, lotions, aqueous or oily suspensions, dispersible powders or granules, emulsions, sprays or aerosols using flowing propellants, like liposomal sprays, nasal sprays etc., douches and suppositories, transdermal patches etc., all for patient-friendly, self-administration of effective amounts of vitamin B₁₂. The invention thereby minimizes inconvenience and discomfort for the patient and alleviates the burden and time demands imposed on medical staff.

Accordingly, the active ingredients of the invention are useful in a method for the prevention or treatment of vitamin B₁₂ disorders in certain combinations. The weight ratio of the respective ingredients may be varied when necessary and will depend upon the effective dose of each ingredient or the effective dose of the combination of all the active ingredients in a formulation. Generally, an effective dose of each will be used. Thus, for example, in a composition of the present invention, the total cobalamin weight in a lozenge will generally be around 3 mg and preferably contains at least three active ingredients in the composition. In a preferred embodiment, a combination of active ingredients is used in the composition, for example, methylcobalamine:hydroxycobalamin:cyanocobalamin. Generally, the ratios of the active ingredients of the invention are in the range of 1:1:2 to 1:1:10. Thus, the amount of methylcobalamine, and hydroxycobalamin advantageously will generally range from 250-750 μg, while the range for cyanocobalamin will generally range from 1500-2500 μg in a 3 mg cobalamin formulation. Other combinations of active ingredients of the present invention are also possible as is understood in the art, and will generally be within the aforementioned range.

The skilled artisan will appreciate that the combination of active ingredients found in the compositions described above also may be administered separately. In addition, the administration of one element may be prior to, concurrent to, or subsequent to the administration of other element(s).

Further, compositions of the present invention may be used in combination with other drugs that are used in the treatment/prevention/suppression or amelioration of vitamin B₁₂ deficiencies or conditions. Such other drugs may be administered, by a route and in an amount commonly used therefore, contemporaneously or sequentially with a compound of the present invention. When a composition of the present invention is used contemporaneously with one or more other drugs or herbal supplements, vitamin supplements, etc., a pharmaceutical composition containing such other drugs in addition to the composition of the present invention is preferred. Accordingly, the pharmaceutical compositions of the present invention include those that also contain one or more other active ingredients, in addition to the compositions of the present invention.

In another embodiment, the present invention provides a kit comprising a composition or formulation of the invention and instructions which comprise for storage, administration, dosing, disease state for which the formulation is useful, etc. In yet another embodiment, the present invention provides an article of manufacture comprising a composition or formulation of the invention and an apparatus to dispense or administer the formulation to a given patient, such as container for housing the compound, etc.

Disorders and Conditions Related to Vitamin B₁₂ Deficiency

Accordingly, the subject compounds are useful in a method for the prevention or treatment of the following diseases, disorders and conditions. The following diseases, disorders and conditions are related to Vitamin B12 deficiency, and therefore may be treated, controlled or in some cases prevented, by treatment with the composition of this invention: (1) pernicious anemia, (2) ataxia, (3) autoimmune disorders, (4) patients receiving long term therapy with gastric acid inhibitors like biguanides, (5) patients with atrophic body gastritis, or have had partial or total gastrectomy, (6) anemia associated with chemotherapy treatment (for example, methotrexate, metformin, phenobarbital, phenytoin, etc. (7) alcohol or substance abuse, (8) inflammation of joints, arthritis, (9) burns, (10) neuro-degenerative disease like Alzeimer's disease, amyotrophic lateral sclerosis or multiple sclerosis, (11) senior dementia, (12) allergic diseases such as rhinitis, allergic asthma, etc., (13) HIV/AIDS where there poor absorption of vitamin B12, (14) irritable bowel syndrome or patients who have undergone intestinal surgery, (15) inflammatory bowel disease, including Crohn's disease and ulcerative colitis, (16) suppression of IgE production, and other disorders where vitamin B12 deficiency is a component.

Modes of Administration

In accordance with the invention, vitamin B₁₂ is instilled in a carrier matrix, such as controlled release lozenges, pills, troches, tablets, hard or soft capsules, syrups or elixirs, pressed pills, gel caps, chewing gum, gels such as metered gels that can be administered intranasally, creams, lotions, aqueous or oily suspensions, dispersible powders or granules, emulsions, sprays or aerosols using flowing propellants, like liposomal sprays, nasal drops, nasal sprays etc., douches and suppositories, transdermal patches etc., all for patient-friendly, self-administration of effective amounts of vitamin B₁₂. The vitamin B₁₂ in formulations such as lozenges, troches, tablets, hard or soft capsules, gum etc. are preferably absorbed directly via the mucosa, such as buccal, nasal mucosa, into the blood stream before being subjected to digestion and degradation in the liver. Preferred vitamin B₁₂ formulations include nasal gels, sublingual lozenges, nasal drops, nasal or pulmonary or other mucosal sprays, fast absorbing capsules or tablets, etc.

Thus, the vitamin B₁₂ formulations of the present invention may be administered, but are not limited to, oral, parenteral (e.g., intramuscular, intraperitoneal, intravenous, ICV, intracistemal injection or infusion, subcutaneous injection, or implant), by inhalation spray, intranasal, transbuccal, mucosal, pulmonary, transdermal, liposomal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

Methods of Making the Compositions

The pharmaceutical compositions for the administration of the compounds of this invention may conveniently be presented in dosage unit form and may be prepared by methods well known in the art of pharmacy. Suitable methods are described in, for example, Remington, The Science and Practice of Pharmacy, ed. Gennaro et al., 20th Ed. (2000), although the skilled artisan will recognize that other methods are known and are suitable for preparing the compositions of the present invention. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active ingredient is included in an effective amount, discussed above, sufficient to produce the desired effect upon the process or condition of diseases.

The pharmaceutical compositions containing the active ingredient may also be in a form suitable for oral use, for example, as tablets. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin; or as soft gelatin capsules wherein the active ingredient is mixed with water or an oil medium, for example peanut oil, liquid paraffin, or olive oil.

Formulations are also useful as dry powders or granules. Dispersible, dry powders are useful for inhalation after aerosolization with a suitable dispersion device. Dry powder dispersion devices for medicaments are described in a number of patent documents. U.S. Pat. No. 3,921,637 describes a manual pump with needles for piercing through a single capsule of powdered medicine. The use of multiple receptacle disks or strips of medication is described in EP 467172 (where a reciprocatable piercing mechanism is used to pierce through opposed surfaces of a blister pack); WO91/02558; WO93/09832; WO94/08522; U.S. Pat. Nos. 4,627,432; 4,811,731; 5,035,237; 5,048,514; 4,446,862; and 3,425,600. Other patents which show puncturing of single medication capsules include U.S. Pat. Nos. 4,338,931; 3,991,761; 4,249,526; 4,069,819; 4,995,385; 4,889,114; and 4,884,565; and EP 469814. WO90/07351 describes a hand-held pump device with a loose powder reservoir. Further dry powder dispensers are also covered in U.S. Pat. No. 6,089,228 which specifically provides an improved apparatus for aerosolizing a powdered medicament, hereby incorporated by reference.

Dispersible powders and granules are also suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may sometimes be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, gels including nasal gels, ointments, jellies, solutions or suspensions, mouth washes and gargles, etc., containing the compounds of the present invention, are employed.

It is the primary object of the present invention to provide patient-friendly modes of delivery to patients of such effective amounts of vitamin B12 analogues without the inconvenience and discomfort of subcutaneous and intramuscular injections.

What is claimed is:

1. A pharmaceutical composition comprising an effective amount of a mixture of methylcobalamine, hydroxycobalamin, and cyanocobalamin and a pharmaceutically acceptable carrier.

2. The composition according to claim 1 wherein said composition is in the form of a gel.

3. The composition according to claim 2 wherein said gel is disposed within a capsule.

4. The composition according to claim 1 wherein said composition is in the form of a sublingual lozenge.

5. The composition according to claim 1 wherein said composition is in the form of a solution suitable for aerosolization for mucosal, nasal or pulmonary administration.

6. The composition according to claim 5 wherein said composition is in the form of a liposomal formulation.

7. The composition according to claim 1 wherein said composition is in the form of fine particles in a form suitable for aerosolization for mucosal or pulmonary administration.

8. The composition according to claim 1 wherein said composition is in the form of a tablet.

9. An apparatus for producing aerosolized doses of a composition according to claim 7, comprising a means for introducing a preselected amount of said composition into a predetermined volume of a flowing propellent to produce an aerosolized quantity of said composition.

10. A method of treating or ameliorating a disease associated with vitamin B₁₂ deficiency, comprising administering to a subject suffering from or suspected of suffering from said disease an effective amount of a composition according to claim 1.

11. The method according to claim 10, wherein said subject suffers from or is suspected to suffer from an anemia.

12. The method according to claim 11, wherein said anemia is pernicious anemia.

13. The method according to claim 11, wherein said anemia is caused by drug administration to said subject, Crohn's disease, and burns.

14. A method of treating or ameliorating a disease or syndrome in a subject, comprising administering to said subject an effective amount of a composition according to claim 1, wherein said disease or syndrome is selected from the group consisting of cobalamin malabsorption, a neurological disorder, atrophic body gastritis, an autoimmune disorder, and symptoms associated with therapy with gastric acid inhibitors or biguanides.

15. The method according to claim 14 wherein said neurological disorder is selected from the group consisting of Alzheimer's disease, amyotrophic lateral sclerosis, multiple sclerosis, and ataxia.

16. A method of treating an inflammatory disease in a subject, comprising administering to said subject an effective amount of a composition according to claim 1.

17. The method according to claim 16 wherein said inflammatory disease is joint inflammation or arthritis.

18. A method of reducing immunoglobulin E production in a subject comprising administering to said subject an effective amount of a composition according to according to claim 1.

19. A method of treating or ameliorating a disease associated with vitamin B₁₂ deficiency, comprising using an apparatus according to claim 9 to administer an effective dose of said composition to a subject suffering from or suspected of suffering from said disease.

20. A kit comprising the pharmaceutical composition of claim 1.
